(19) United States
(12) Patent Application Publication
(10) Pub. No.: US 2002/0077589 A1
(43) Pub. Date: Jun. 20, 2002

(54) Method and apparatus for producing an injectable foam

(76) Inventor: Lorenzo Tessari, Verona-Peschiera (IT)

Correspondence Address: Joseph C. Sullivan, Pitney, Hardin, Kipp & Szuch LLP, 711 Third Avenue, New York, NY, 10017, US

(21) Appl. No.: 09/767,258

(22) Filed: Jan. 22, 2001

(30) Foreign Application Priority Data
Dec. 18, 2000 (GB) 00 30858.5

(51) Int. Cl. A61M 37/00

(52) U.S. Cl. 604/82

(57) ABSTRACT

A method of producing injectable foam, comprises: providing a first syringe containing a gas and a second syringe containing a liquid comprising a foaming substance and passing the gas and the liquid back and forth between the first syringe and the second syringe by operation of the first syringe and the second syringe so that the gas and the liquid mix turbulently to form the foam.

The invention relates to a method and apparatus for producing an injectable foam.

Injection of a sclerosant is an alternative to surgery in the treatment of varicose veins. Injection of liquid sclerosant can be used to treat smaller varicosities but it is known that injection of the sclerosant as a foam rather than a liquid is more effective in treating larger varicosities.

Known methods of producing injectable sclerosant foam include agitation of a container containing a foamable sclerosing agent. European Patent No. 0656203 describes production of a sclerosing micro-foam by mechanical beating of a sclerosing solution, contained in a sterile hermetic container, with a brush rotated at between 8000 rpm and 15000 rpm by a micro-motor, the container optionally being connected to a pressure bottle of oxygen or other physiological gas. That method thus requires rather complicated equipment.

It is an aim of the invention to produce an apparatus and method that avoids or mitigates that disadvantage and which, in particular, provides a simple means of producing injectable sclerosant foam.

According to an aspect of the invention, there is provided a method of producing injectable foam, comprising: providing a first syringe containing a gas and a second syringe containing a liquid comprising a foaming substance and passing the gas and the liquid back and forth between the first syringe and the second syringe by operation of the first syringe and the second syringe so that the gas and the liquid mix turbulently to form the foam.

Foams arise in the presence of a gas, a liquid, a foaming substance and turbulence. The foaming substance may be a surfactant or any other substance causing a foam to be produced. Turbulence will naturally arise as a consequence of the back-and-forth motion between the syringes. Preferably, the liquid comprises a sclerosant . Examples of sclerosants suitable for forming foams include surfactants such as sodium tetradecylsulphate (STS), polidocanol, ethanolamine oleate and sodium morrhuate. The sclerosant may be diluted for example with sterile water or saline. With suitable modification, other sclerosants such as hypertonic glucose or gluco-saline solutions, chromic glycerol and iodic solutions may also be used.

Suitable gases are, for example, air, oxygen, carbon dioxide, helium or mixtures of such gases. Non-invasive, physiological gases should especially be mentioned. Preferably, the method includes a preliminary step of at least partially filling the first syringe with the gas. The gas may be sterilised before or after the first syringe is at least partially filled. Sterilisation may be brought about, for example, by exposure to y-radiation.

Preferably, the first and second syringes are attached to a valve that is selectively adjustable between a first position in which the first and second syringes are not in communication with each other and a second position in which the first and second syringes are in communication with each other. That provides a convenient means to keep the gas and the sclerosant liquid separate, by keeping the valve in the first position, until it is desired to mix them, when the valve may be adjusted to the second position. The valve may be a three-port valve. Preferably, the valve is attached to the first syringe prior to attachment of the second syringe. Preferably, the valve retains in the first syringe the gas contained in the first syringe. Preferably, the second syringe is at least partially filled when the valve is in the first position. The second syringe may be at least partially filled through a nozzle with which it is in communication when the valve is in the first position. Preferably the nozzle is attached to a needle.

Alternatively, the method may include a preliminary step of at least partially filling the second syringe with the liquid.

When the foam has been produced, it may be withdrawn into either syringe, which syringe may then be detached from the valve. A needle may then be connected to it so that it is ready for use in an injection. The foam is thus produced in a syringe that may be used directly, without the need to transfer it to another container, a transfer that might result in deterioration and/or contamination of the foam.

Excess foam may be retained in one syringe after the other syringe is detached. It is thus retained in a sterile environment and is ready for use at a later time. The back and forth transfers may need to be repeated to return the foam to a useable state.

The method may further comprise the step of adjusting the valve to a third position in which the first syringe and the second syringe are in reduced communication compared with the second position. Reducing the communication between the syringes has been found to produce improved foam, that is foam having smaller bubbles and a longer life. The valve may comprise a collar member defining at least two apertures and a valve member defining a bore and communication between the first and second syringes may be reduced by rotation of the collar member so that the apertures and bore are misaligned relative to each other. Communication may be progressively reduced further by adjusting the valve to a number of further positions.

Some or all of the passages of liquid back and forth may be carried out with the apertures and bore misaligned.

Preferably the air and liquid are passed back and forth at least five times. More preferably, the air and liquid are passed back and forth at least ten times. Still more preferably, the air and liquid are passed back and forth at least twenty times.

Preferably, the capacities of the first and second syringes are in the range 1 ml to 20 ml. More preferably, the capacities are in the range 2 ml to 5 ml. Preferably, the first syringe has a larger capacity than the second syringe.

Preferably the ratio of the volume of gas to sclerosant liquid is from 2:1 to 8:1. More preferably, the ratio of volume of gas to sclerosant is from 4:1 to 6:1. Still more preferably, the ratio of the volume of gas to sclerosant is 5:1.

Preferably, the concentration of the sclerosant is in the range 0.1% to 5% by weight. Concentrations of STS in the range 1% to 3% by weight have been found to be suitable in producing foam for treatment of saphenous and recurrent varicoses. Concentrations of STS in the range 0.3% to 1% by weight have been found to be particularly suitable in producing foam suitable for treatment of collateral varicose veins.

Preferably, the dose of the sclerosant liquid in the second syringe is in the range 0.25 ml to 4 ml. Doses depend on length and calibre of veins to be treated with the foam produced by the method. Doses of foam in the range 1 ml to 10 ml have been found to be suitable in producing foam for treatment of saphenous and recurrent varicoses; we have found that 2 ml to 4 ml is particularly effective. We have found that doses of foam in the range of 1 ml to 2 ml are particularly suitable in producing foam suitable for treatment of collateral varicose veins.

It is a particular advantage that the foam persists for sufficiently long that it can be injected and have effect but it does not persist for too long in the body.

The foam produced by the method according to the invention is suitable for use in methods of treatment of, for example, long and short sapheneous veins, recurrent varices and collaterals, reticular varices, telangiectasias, varicocele recurrences or perforator incompetence. A foam produced according to the invention may have other uses in medicine. It may be suitable for use in methods relating to, for example, phlebology, oesophageal varices, proctology or angiology.

According to another aspect of the invention there is also provided a method of treating varicose veins by injection with an effective amount of sclerosant foam produced by: providing a first syringe containing a gas; providing a second syringe containing a sclerosant liquid; and passing the gas and the sclerosant back and forth between the first syringe and the second syringe so that the gas and the liquid mix turbulently to form the foam.

According to another aspect of the invention, there is also provided a method of treating oesophageal varices by injection with an effective amount of sclerosant foam produced by: providing a first syringe containing a gas and a second syringe containing a sclerosant liquid comprising a surfactant and passing the gas and the liquid back and forth between the first syringe and the second syringe by operation of the first syringe and the second syringe so that the gas and the liquid mix turbulently to form the foam.

According to another aspect of the invention there is also provided a method of treating haemorrhoids by injection with an effective amount of a sclerosant foam produced by: providing a first syringe containing a gas and a second syringe containing a sclerosant liquid comprising a surfactant and passing the gas and the liquid back and forth between the first syringe and the second syringe by operation of the first syringe and the second syringe so that the gas and the liquid mix turbulently to form the foam.

According to another aspect of the invention there is also provided a method of treating varicocele by injection with an effective amount of a sclerosant foam produced by: providing a first syringe containing a gas and a second syringe containing a sclerosant liquid comprising a surfactant and passing the gas and the liquid back and forth between the first syringe and the second syringe by operation of the first syringe and the second syringe so that the gas and the liquid mix turbulently to form the foam.

The use of foam is particularly advantageous in methods of treatment with co-use of ultrasound, such as duplex guiding, and in diagnosis by ultrasound because foam is echogenic.

According to another aspect of the invention there is provided apparatus comprising a first syringe, a second syringe and a valve to which the first and second syringes are attached and which is selectively adjustable between a first position in which the first and second syringes are not in communication with each other and a second position in which the first and second syringes are in communication with each other.

Preferably, the valve is selectively adjustable between the first and second positions and a third position, in which the first syringe and the second syringe are in reduced communication compared with the second position. More preferably, the valve is adjustable to a number of further positions and the communication can thereby be progressively further reduced. The valve may comprise a collar member defining at least two apertures and a valve member defining a bore so that communication between the first and second syringes is reducible by rotation of the collar member and/or the valve member so that the apertures and bore are misaligned relative to each other. Preferably, the first syringe contains a gas. Preferably, the second syringe contains a liquid comprising a foaming substance. More preferably the liquid comprises a sclerosant.

Preferably, the sclerosant is purified sodium tetradecylsulfate. The gas may be, for example, sterile air or sterile CO₂.

Preferably, the valve is a three-port valve. More preferably, the valve is a T-shaped rotary valve.

The first and second syringes may be attached to the valve by a screw-thread.

Preferably, the second syringe can be filled through a nozzle with which it is in communication when the valve is in the first position. More preferably, the nozzle is attached to a needle.

The capacities of the first and second syringes may be in the range 1 ml to 20 ml. Preferably, the first syringe has a larger capacity than the second syringe. Preferably, the ratio of the volume of gas to sclerosant liquid is in the range of from 2:1 to 8:1. Preferably, the concentration of the sclerosant is in the range 0.1% to 4% by weight. Preferably, the dose of the sclerosant liquid with which the second syringe is filled is in the range 0.25 ml to 4 ml.

According to another aspect of the invention there is provided a kit comprising a first syringe, a second syringe and a valve to which the first and second syringes may be attached and which is selectively adjustable between a first position, in which the first and second syringes are not in communication with each other, and a second position, in which the first and second syringes would be in communication with each other if they were attached to the valve.

Preferably, in the kit the valve is attached to the first syringe and is in the first position and the first syringe contains a sterilised gas. If desired, the kit further comprises a liquid sclerosant, contained, for example, in the second syringe.

According to another aspect of the invention there is provided apparatus for producing an injectable foam comprising a 3-way valve having a first valve port and a second valve port and a first syringe connected to the first valve port and containing a sterile gas, wherein the valve is capable of providing restricted communication between the first and the second ports.

Preferably, the apparatus further comprises a second syringe connected to the second valve port.

According to another aspect of the invention there is provided an injectable sclerosant foam produced by: drawing a gas into a first syringe; drawing a sclerosant liquid into a second syringe; and passing the gas and the sclerosant back and forth between the first syringe and the second syringe by operation of the first syringe and the second syringe so that the gas and the liquid mix turbulently to form the foam.

An illustrative embodiment of the invention will now be described in detail with reference to the drawings, in which:

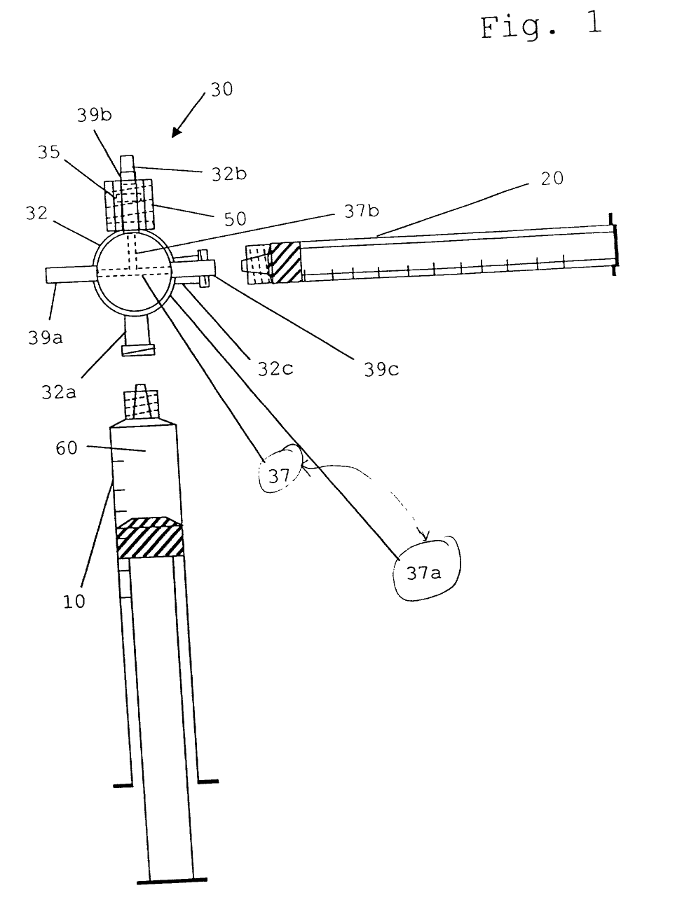

FIG. 1 is a schematic representation of components of the apparatus, according to the invention, for producing injectable sclerosant foam.

FIGS. 2 to 6 are schematic depictions of the apparatus at various stages in the production of injectable foam by a method according to the invention.

In an embodiment of the invention, two sterile plastic syringes 10, 20 are provided, together with a valve 30 (FIG. 1). Syringe 10 has a capacity of 6 ml and is filled with approximately 2.5 ml of air 60, which has been sterilised by 2.5 Mrd Gamma irradiation. Each syringe is 10, 20 is a Luer syringe, which is lockable to the valve 30 by a screw thread at the nozzle of the syringe 10, 20.

In a kit provided to the user, the air 60 is retained in syringe 10 by a plastic three-way valve 30 (FIG. 2). The valve 30 has an outer plastic cylindrical collar 32 having a first projecting, partially threaded, tubular port 32a, to which syringe 10 is screwed, forming an air-tight seal. Collar 32 also has a second, projecting, tubular port 32b, which is arranged opposite port 32a, and can retain a sterile needle, by means of threaded collar 50, which is in turn retained by flange 35 on port 32b. Collar 32 also has a third, projecting, tubular port 32c, which is arranged at 90 degrees to ports 32a, b, forming a T-shape arrangement, and is partially threaded for retention of syringe 20. Valve 30 has an inner plastic cylindrical valve member 37, retained within the collar 32 and having bore 37a extending along a diameter. Member 37 also has a bore 37b, which extends along half a diameter and thus intersects at right angles bore 37a, with which it is in communication, forming a T-shaped bore with it. The bores 37a, 37b have an internal diameter of the order of 1 mm. Member 37 has integral arms 39a , b, c which project beyond collar 32 to form a T-shaped manually rotatable means that serves to enable rotation of member 37 and to indicate the orientation of bores 37a, b therein.

Member 37 is rotatable into various positions. It is positionable so that bores 37a, b are in communication with ports 32a, b, c, which are hence in communication with each other. It is rotatable from that position through 90 degrees so that ports 32a and 32c are in communication with each other and port 32b is sealed off from each of them (Position B1). It is rotatable by a further 90 degrees so that ports 32a and 32b are in communication with each other via bore 37a only and port 32c is sealed off from each of them. It is rotatable by a further 90 degrees so that ports 32b and 32c are in communication with each other and port 32a is sealed off from each of them (Position A). Member 37 is also rotatable into various intermediate positions in which the ends of bores 37a, b do not coincide with any of ports 32a, b, c, so that each of the ports is sealed off from each other port. Member 37 is also rotatable into positions in which at least one of bores 37a, b is slightly misaligned from at least two of ports 32a, b, c, so that the apertures formed at the ends of the bores are smaller than those formed when the bores are coaxial with their respective port or ports. For example, Member 37 can be rotated slightly from Position B1 so that port 32a and port 32c are in reduced communication with each other (Position B2). In position B2, the area of the communicating aperture between port 32a and bore 37a is reduced to about 25% of the area of that aperture in the B1 position.

Syringe 20 has a capacity of 3 ml and its plunger is initially fully depressed so that it is empty.

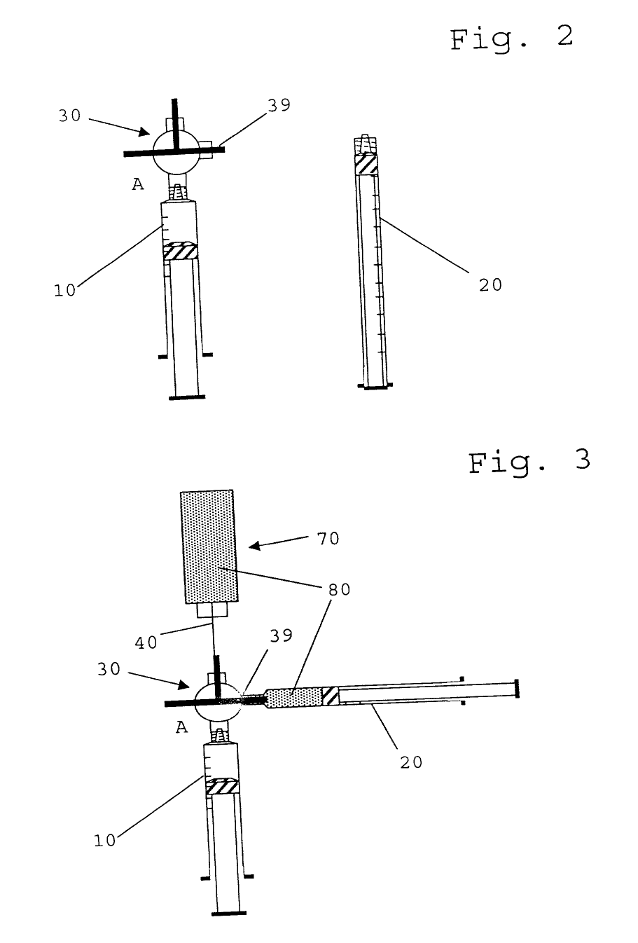

At the beginning of an example of a method according to the invention (FIG. 2), member 37 is in position A so that port 32b and port 32c are in communication with each other and port 32a is sealed, retaining 2.5 ml sterile air 60 in syringe 10. Syringe 20 is then screwed to port 32c and sterile needle 40 is attached to port 32b (FIG. 3). Needle 40 is inserted into an ampule or vial 70 containing a liquid sclerosant 80 (Fibro-vein 0.2%, 0.5%, 1% or 3%, being Sodium Tetradecyl Sulphate Injection BP Standard) and 0.5 ml sclerosant (or another desired amount) is withdrawn into syringe 20.

Member 37 is then rotated into position B1 (FIG. 4), so that syringe 10 and syringe 20 are in communication with each other. At this stage it should be checked that the Luer Lock couplings between the syringes and the 3-way valve are tight.

The operator then takes one of syringes 10, 20 into each hand and depresses the plunger of syringe 10 so that air is transferred into syringe 20. The plunger of syringe 20 is then depressed and air and sclerosant are transferred back into syringe 10. The plungers are rapidly alternately depressed to transfer sclerosant back and forth between the syringes 10, 20. The rapid motion results in turbulent flow, which leads to production of foam 100 from the mixing of the air and the surfactant sclerosant.

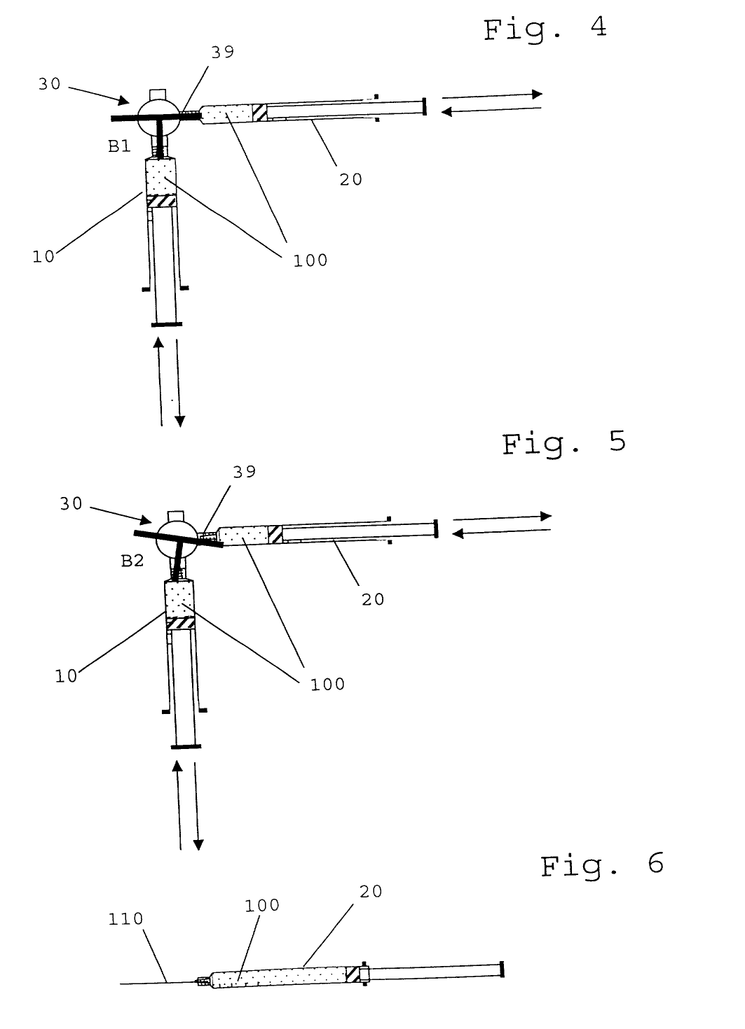

In order to improve the quality of the foam 100 (that is, to produce a foam having smaller bubbles and a longer life), the member 37 is now rotated to position B2 (FIG. 5), so that the aperture between port 32a and bore 37a (and that between port 32c and bore 37b) is reduced, being partially blocked by member 37 and collar 32. Rapid alternation of the plungers is continued. In one trial, twenty passages (i.e. ten complete passages from one syringe back to itself) were sufficient to produce foam that was of good quality; that is, it maintained its nature for 4 minutes to 5 minutes and then began to liquefy visibly.

The quality of foam produced will vary depending on the size of the aperture between ports 32a, c and bores 37a, b (i.e., on the relative positions of member 37 and collar 32) on whether the size of the aperture is varied during the method. Using purified sodium tetradecylsulphate with the apparatus described, it has been found that best foam is produced by keeping member 37 in position B2 throughout production of the foam. Experimentation to determine the exact optimal member position is advised for any particular combination of, for example, sclerosant, syringe size, gas or valve bore size.

The quantities given above have been found to be sufficient to produce 3 ml of foam 100. A sufficient quantity of foam for injection is next withdrawn into syringe 20. Syringe 20 is then disconnected from valve 30 and a sterile injection needle 110 is attached to syringe 20 (FIG. 6). The foam is then ready for injection from syringe 20.

Excess foam 100 is retained in syringe 10, which is sealed by rotation of member 32. It may be necessary to repeat alternate depression of the plungers if there is a delay between generation and use of the foam. It is important that the foam is thick and viscous immediately prior to injection. Typical bubble sizes suitable for use are up to 100 μm. Bubbles in the range 40 μm to 100 μm should especially be mentioned and bubbles in that range were produced in this embodiment when the member 37 was kept in the B1 position throughout foam production.

It is important to note that the use of foam appears to potentiate the concentration of sodium tetradecyl sulphate. The use of foam in treating small veins is not generally recommended and should only be carried out with caution and by using a weaker concentration of sclerosant than would be used when treating such veins conventionally.

What is claimed is:

1. A method of producing injectable foam, comprising: providing a first syringe containing a gas and a second syringe containing a liquid comprising a foaming substance and passing the gas and the liquid back and forth between the first syringe and the second syringe by operation of the first syringe and the second syringe so that the gas and the liquid mix turbulently to form the foam.

2. A method as claimed in claim 1, in which the liquid comprises a sclerosant.

3. A method as claimed in claim 1, including a preliminary step of at least partially filling the first syringe with the gas.

4. A method as claimed in claim 3, in which the gas is sterilised before the first syringe is at least partially filled.

5. A method as claimed in claim 1, in which the first and second syringes are attached to a valve that is selectively adjustable between a first position in which the first and second syringes are not in communication with each other and a second position in which the first and second syringes are in communication with each other.

6. A method as claimed in claim 5, in which the valve is attached to the first syringe prior to attachment of the second syringe.

7. A method as claimed in claim 5, in which the second syringe is at least partially filled when the valve is in the first position.

8. A method as claimed in claim 7, in which the second syringe is at least partially filled through a nozzle with which it is in communication when the valve is in the first position.

9. A method as claimed in claim 1, including a preliminary step of at least partially filling the second syringe with the liquid.

10. A method as claimed in claim 5, in which, when the foam has been produced, it is withdrawn into the second syringe, which is then detached from the valve.

11. A method as claimed in claim 10, in which a needle is then connected to the second syringe so that it is ready for use in an injection.

12. A method as claimed in claim 10, in which excess foam is retained in the first syringe after the second syringe is detached.

13. A method as claimed in claim 5, further comprising a step of adjusting the valve to a third position in which the first syringe and the second syringe are in reduced communication compared with the second position.

14. A method as claimed in claim 13, in which the valve comprises a collar member defining at least two apertures and a valve member defining a bore and communication between the first and second syringes is reduced by rotation of the collar member and/or the valve member so that the apertures and bore are misaligned relative to each other.

15. A method as claimed in claim 13, in which communication is progressively reduced further by adjusting the valve to a number of further positions.

16. A method as claimed in claim 1, in which the air and liquid are passed back and forth at least five times.

17. Apparatus comprising a first syringe, a second syringe and a valve to which the first and second syringes are attached and which is selectively adjustable between a first position in which the first and second syringes are not in communication with each other and a second position in which the first and second syringes are in communication with each other.

18. Apparatus according to claim 17, in which the first syringe contains a gas.

19. Apparatus according to claim 17, in which the second syringe contains a liquid comprising a foaming substance.

20. Apparatus according to claim 19, in which the liquid comprises a sclerosant.

21. Apparatus as claimed in claim 20, in which the sclerosant is purified sodium tetradecylsulfate.

22. Apparatus as claimed in claim 18, in which the gas is sterile air.

23. Apparatus as claimed in claim 18, in which the gas is sterile CO₂.

24. Apparatus according to claim 17, in which the valve is selectively adjustable between the first and second positions and a third position, in which the first syringe and the second syringe are in reduced communication compared with the second position.

25. Apparatus as claimed in claim 24, in which the valve is adjustable to a number of further positions and the communication can thereby be progressively further reduced.

26. Apparatus as claimed in claim 17, in which the valve comprises a collar member defining at least two apertures and a valve member defining a bore and communication between the first and second syringes is reducible by rotation of the collar member and/or the valve member so that the apertures and bore are misaligned relative to each other.

27. Apparatus as claimed in claim 17, in which the valve is a three-port valve.

28. Apparatus as claimed in claim 27, in which the valve is a T-shaped rotary valve.

29. Apparatus as claimed in claim 17, in which at least one of the first and second syringes is attached to the valve by a screw-thread.

30. Apparatus as claimed in claim 17, in which the second syringe can be filled through a nozzle with which it is in communication when the valve is in the first position.

31. Apparatus as claimed in claim 30, in which a needle is attached to the nozzle.

32. Apparatus as claimed in claim 17, in which the capacities of the first and second syringes are in the range 1 ml to 20 ml.

33. Apparatus as claimed in claim 17, in which the first syringe has a larger capacity than the second syringe.

34. Apparatus as claimed in claim 17, in which the ratio of the volume of gas to sclerosant liquid is in the range of from 2:1 to 8:1.

35. Apparatus as claimed in claim 17, in which the concentration of the sclerosant is in the range 0.1% to 4% by weight.

36. Apparatus as claimed in claim 17, in which the dose of the sclerosant liquid with which the second syringe is filled is in the range 0.25 ml to 4 ml.

37. A kit comprising a first syringe, a second syringe and a valve to which the first and second syringes can be attached and which is selectively adjustable between a first position, in which the first and second syringes are not in communication with each other, and a second position, in which the first and second syringes would be in communication with each other if they were attached to the valve.

38. A kit as claimed in claim 37, in which the valve is attached to the first syringe and is in the first position and the first syringe contains a sterilised gas.

39. A kit as claimed in claim 38, further comprising a liquid sclerosant.

40. A kit as claimed in claim 39, in which the sclerosant is contained in the second syringe.

41. Apparatus for producing an injectable foam comprising a 3-way valve having a first valve port and a second valve port and a first syringe connected to the first valve port and containing a sterile gas, wherein the valve is capable of providing restricted communication between the first and the second ports.

42. Apparatus as claimed in claim 41, further comprising a second syringe connected to the second valve port.

43. An injectable sclerosant foam produced by: drawing a gas into a first syringe; drawing a sclerosant liquid into a second syringe; and passing the gas and the sclerosant back and forth between the first syringe and the second syringe by operation of the first syringe and the second syringe so that the gas and the liquid mix turbulently to form the foam.

44. A method of treating varicose veins by injection with an effective amount of a sclerosant foam produced by: providing a first syringe containing a gas and a second syringe containing a sclerosant liquid comprising a surfactant and passing the gas and the liquid back and forth between the first syringe and the second syringe by operation of the first syringe and the second syringe so that the gas and the liquid mix turbulently to form the foam.

45. A method of treating oesophageal varices by injection with an effective amount of a sclerosant foam produced by: providing a first syringe containing a gas and a second syringe containing a sclerosant liquid comprising a surfactant and passing the gas and the liquid back and forth between the first syringe and the second syringe by operation of the first syringe and the second syringe so that the gas and the liquid mix turbulently to form the foam.

46. A method of treating haemorrhoids by injection with an effective amount of a sclerosant foam produced by: providing a first syringe containing a gas and a second syringe containing a sclerosant liquid comprising a surfactant and passing the gas and the liquid back and forth between the first syringe and the second syringe by operation of the first syringe and the second syringe so that the gas and the liquid mix turbulently to form the foam.

47. A method of treating varicocele by injection with an effective amount of a sclerosant foam produced by: providing a first syringe containing a gas and a second syringe containing a sclerosant liquid comprising a surfactant and passing the gas and the liquid back and forth between the first syringe and the second syringe by operation of the first syringe and the second syringe so that the gas and the liquid mix turbulently to form the foam.
